(19) United States
(12) Patent Application Publication
(10) Pub. No.: US 2015/0087926 A1
(43) Pub. Date: Mar. 26, 2015

(54) System and Method for Facilitating Remote Medical Diagnosis and Consultation

(71) Applicants: Nir Raz, Had HaSharon (IL); Shiri Raz, Hod Hasharon (IL)

(72) Inventors: Nir Raz, Had HaSharon (IL); Shiri Raz, Hod Hasharon (IL)

(21) Appl. No.: 14/386,004

(22) PCT Filed: Apr. 16, 2013
(86) PCT No.: PCT/IL2013/050327

§ 371 Date: Sep. 18, 2014

Related U.S. Application Data
(60) Provisional application No. 61/635,302, filed on Apr. 19, 2012.

(51) Int. Cl. A61B 5/00; A61B 13/00; A61B 7/04; A61B 5/01; A61B 1/227

(52) U.S. Cl. CPC A61B 5/6898; A61B 5/01; A61B 1/227; A61B 7/04; A61B 13/00; A61B 5/0077; A61B 5/0013; A61B 5/7282; A61B 5/0022
USPC 600/301

(57) ABSTRACT

The present invention is a method and system for facilitating a remote preliminary medical diagnosis of patients. An examination apparatus is provided comprising a single jacket attached to a smartphone and enabling a set of necessary examinations for preliminary medical diagnosis. In another aspect of the present invention the apparatus consists of a single attachment connected to the smart phone. The invention further discloses methods for remotely performing a set of necessary examinations by which a physician makes a remote diagnosis of the patient and if required issues a digital prescription or summons the patient for further examination. The medical information can be recorded, filed and shared by the patient, by the physician and by the pharmacy.

### BACKGROUND OF THE INVENTION

1. Technical Field

The present invention relates to a system and method for facilitating remote medical diagnosis and consultation, more particularly but not exclusively, the present invention relates to preliminary medical diagnosis and consultation of patients and to a system and method to manage such diagnosis.

2. Description of the Related Art

In traditional health care system, medical diagnosis and consultation is conducted face to face between the patients and the doctors in the clinics and hospitals. The traditional health care system requires patients to visit a clinic or a hospital for diagnosis. The visit to a clinic or a hospital may in itself pose a risk of infection due to the physical proximity of the patient to other patients at the medical facility (at the waiting rooms for example). Even when trying to avoid physical proximity to other patients, diseases may spread by other means such as the ventilation systems. In addition, traveling to and from the medical facilities is time consuming, and inconvenient especially when feeling ill. As for the medical facilities, they too require tedious procedures to process the visit of each patient arriving at the facility. The aforementioned drawbacks are stressed especially when the patients are children. As children need to be driven to and from the medical facilities by their parents, each visit poses the aforementioned risks and inconvenience both on the child and on the parent.

Other differences are taken into consideration when treating children. For instance, children's immune system is considered more vulnerable than the immune system of adults. As a result, children are ill more often and usually require frequent medical attention. In addition, when treating children, it can be more difficult to diagnose the condition of the child as some children are too young to adequately express their symptoms to the physician and to answer his questions. Many physicians require the diagnosis of: the ears, the throat, the breathing and the heart, whenever a child attends the clinic. These checkups are essential to determine the overall condition of the child and are therefore considered as the “Preliminary Diagnosis”. Other checkups may also be performed in addition to the Preliminary Diagnosis such as: body temperature, eyes condition etc., as well as other designated checkups which are subject to the specific symptoms of the child. In other cases, the Preliminary Diagnosis is sufficient and additional checkups are not required. The Preliminary Diagnosis may suffice to determine the medical condition of the patient and the treatment which is to be provided. In other cases, the Preliminary Diagnosis may suffice to determine the referral of the patient to a specialist.

The health care industry is undergoing an evolution by integrating Information Technology (IT). One such application of the Information Technology facilitates remote medical diagnosis and consultation. A technology-based health care system that fully integrates the technical and social aspects of patient care and therapy should be able to flawlessly connect the client with care providers irrespective of separation distance or location of the participants. While clinicians will continue to treat patients in accordance with accepted modem medical practice, developments in communications technology make it ever more possible to provide a seamless system of remote patient diagnostics, care and medical services in a time and place independent manner. There exist many systems and methods with the objective of facilitating remote medical diagnosis and consultation. Some examples of such systems and methods are described as follows.

International Application No. WO2009/094132 to Winnik discloses a method of diagnosing an ear infection, the method comprising imaging a patient's ear drum using an otoscope sensor, wirelessly transmitting the image of the ear drum from the sensor to a first programmable communication device, then transmitting the image of the ear drum from the first programmable communication device to a remote base unit, and finally, analyzing the image to determine a course of treatment.

International Application No. PCT/US2003/038603 to Ineedmd.com Inc. discloses a method for remote medical consulting which includes collecting diagnostic data using at least one wearable device contoured to at least a portion of a person's hand, transmitting the diagnostic data to a remote location, transmitting audio data and video images of the patient to the remote location, and communicating diagnosis and/or treatment information to the patient based at least in part on the diagnostic data. The treatment information may include a prescription electronically transmitted to the patient or a pharmacy. The method includes billing of the patient via credit or debit card, bank account, or a third party, such as an insurance company. The diagnostic data as well as the audio and video data may be transmitted wirelessly via cellular or satellite communication networks and/or using a wide area computer network such as the internet.

However, the above described example does not cover remote preliminary medical examination consisting of several essential checkups.

It would be desirable to provide a method and system to facilitate remote Preliminary Diagnosis and consultation.

It would be desirable to provide a single apparatus which can be attached to a smart phone, to provide Preliminary Diagnosis.

It would be desirable to provide a method and system for remote preliminary medical examination that saves visits to medical facilities and other associated facilities.

It would be desirable to provide a system and method to record, file and share the diagnosed information both by the patient as well as by the physician, the medical facilities and other associated facilities.

The advantages of the present invention will become apparent from the following description, taken in connection with the accompanying drawings, wherein, by way of illustration and example, an embodiment of the present invention is disclosed.

### SUMMARY OF THE INVENTION

In accordance with the first aspect of the present invention, a system for facilitating remote medical diagnosis of a patient is provided. The system may comprises an examination apparatus which is a Preliminary Diagnosis jacket attached to a smartphone and operated by a dedicated smart phone application, a computer network in communication with the examination apparatus via a wireless communication link and a server located remotely. During the examination the patient activates the examination apparatus which will perform an examination of the patient. The examination apparatus collects the examination data during the examination and transmits the examination data over a network to a server to which a physician has access. Based on the examination data, the physician can review the patient's health condition and offer an appropriate diagnosis for the patient. The jacket may be designed to enable capturing the required information for an adequate preliminary examination and the physician may view the captured information on line and instruct the patients as of how to operate the apparatus.

A second aspect is the Preliminary Diagnosis apparatus which can be attached to a smart phone, by a cable or a wireless link The Preliminary Diagnosis apparatus comprises an examination module which includes an ergonomic grip, a microphone, an illumination means, an infra-red sensor, an optical arrangement and a medical device connector. The attachment may be designed to enable capturing the required information for an adequate preliminary examination.

In accordance with a third aspect of the present invention, a method for facilitating remote medical diagnosis of a patient is provided. This method comprises the following steps: registering with the medical facility to schedule a remote examination with a physician, receiving a call by the physician as scheduled, attaching the Preliminary Diagnosis apparatus to the smart phone, a patient securely logs in into a network, an examination of the patient is conducted and the examination data (video, audio, pictures, or any other data) is send over the network to the physician. The physician makes a diagnosis of the patient and, if needed, issues a medical prescription and/or directs the patient for further examination/treatment at the hospital or clinic.

The method of present invention may also include receiving instructions as how to operate the examination apparatus to obtain information sufficient to a Preliminary

Diagnosis, recording the captured information, saving and archiving the records by the system, enabling sharing with pharmacies\specialists and sending digital prescriptions and diagnosis reports .

### BRIEF DESCRIPTION OF THE DRAWINGS

Exemplary embodiments of the present invention will now be described, by way of example only, with reference to the accompanying drawings, in which:

FIG. 1A is a perspective view of an embodiment of the all in one attachment examination apparatus of the present invention.

FIG. 1B is a perspective view of an embodiment of the examination apparatus of the present invention, showing a user interface.

FIG. 2 is a perspective view of the all in one jacket apparatus of the present invention.

FIG. 3 is a perspective view of yet another embodiment of the examination apparatus of the present invention.

FIG. 4 is a perspective view of the examination apparatus of the present invention used as a stethoscope.

FIG. 5 is a perspective view of the examination apparatus of the present invention used as a thermometer.

FIG. 6 is a schematic diagram of the remote diagnosis system of the present invention.

FIG. 7 is a flowchart of an exemplary method for remote examination and diagnosis, according to an embodiment of the present invention.

FIG. 8 and FIG. 9 schematically show perspective views of a jacket configured to be attached to a standard smart phone for performing the preliminary remote medical examination.

FIG. 10 schematically shows a perspective view of a nozzle designed to capture images inside the ear.

### DETAILED DESCRIPTION

In the following detailed description of the invention, numerous specific details are set forth in order to provide a thorough understanding of the invention. However, it will be obvious to one skilled in the art that the invention may be practiced without these specific details. In other instances well known methods, procedures, components, and elements have not been described in detail so as not to unnecessarily obscure aspects of the invention.

It will be readily understood that the components of the present invention, as generally described and illustrated in the figures, may be arranged and designed in a wide variety of different configurations. Thus, the following more detailed description of the embodiments of the apparatus and methods of the present invention, as represented in the figures, is not intended to limit the scope of the invention, but is merely representative of selected embodiments of the invention.

In one embodiment, a system for facilitating a remote medical diagnosis is provided that includes a smartphone and a medical device configured to be connected to the smartphone. The smartphone together with the medical device is used as an examination apparatus and the examination itself and/or its results are transmitted to the physician in order to perform a medical diagnosis of the examined patient.

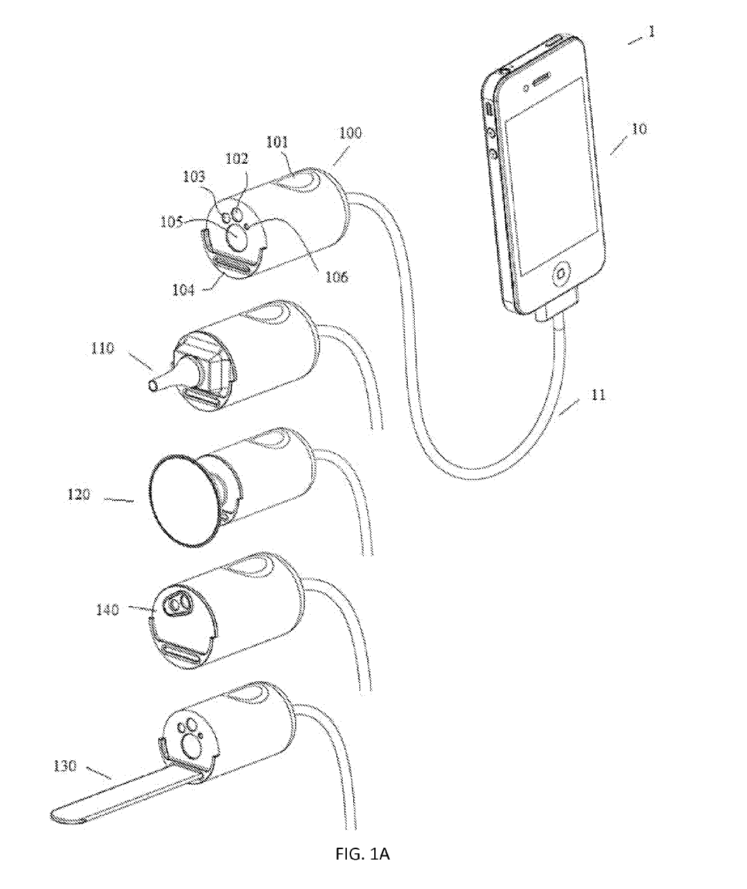

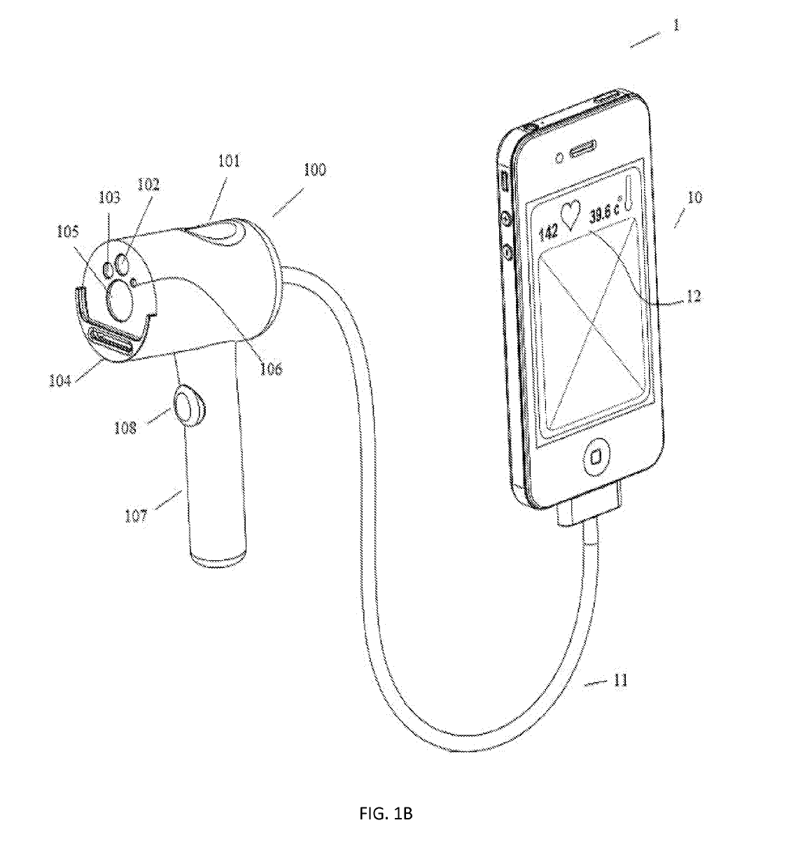

In the embodiment shown in the FIG. 1A it can be seen that an examination apparatus 1 comprises a smartphone 10 and an all in one examination module 100 which is connected to the smartphone through a wireless connection (such as Wi-Fi or Bluetooth) or through a data cable 11. The examination module 100 includes an ergonomic grip 101, a microphone 106, an illumination means 102, an infra-red sensor 103, an optical arrangement 105 and a medical device connector 104. When a speculum 110 is connected to the medical device connector 104, the examination apparatus 1 can be used as an otoscope. When a diaphragm 120 is connected to the medical device connector 104, the examination apparatus 1 can be used as a stethoscope. An infra-red filter 140 is used with the examination module 100 when an examination apparatus 1 is used as a thermometer. Connecting a tongue depressor 130 to the medical device connector 104 facilitates capturing clear images from the throat/mouth to enable reliable diagnosis by the physician and\or by medical analysis software.

FIG. 1B shows a user interface 12 displayed on the screen of the smartphone 10. The user interface 12 may include a video window and a display for showing the examination data and can display instruction as of how to perform the examination. The user interface 12 can also be based on a smart phone application incorporating a game to pacify children patients while they are being examined without disrupting the examination process. The examination module 100 may include a handle 107 with a multi-purpose switch 108 which is used as on/off switch and also may be used for taking snap pictures from the video stream transmitted by the examination module 100 to the smartphone 10. The examination module 100 may also include a rechargeable/non-rechargeable battery. The device map be operated by the patient himself in accordance with instructions given online by the physician, the device can also be operated by a parent of a child or by a caregiver of a patient. Yet alternatively, such devices can be operated directly by doctors and nurses at their clinics to ease the documentation process of the diagnosis by the system, or at remote locations where medical equipment and facilities are scarce.

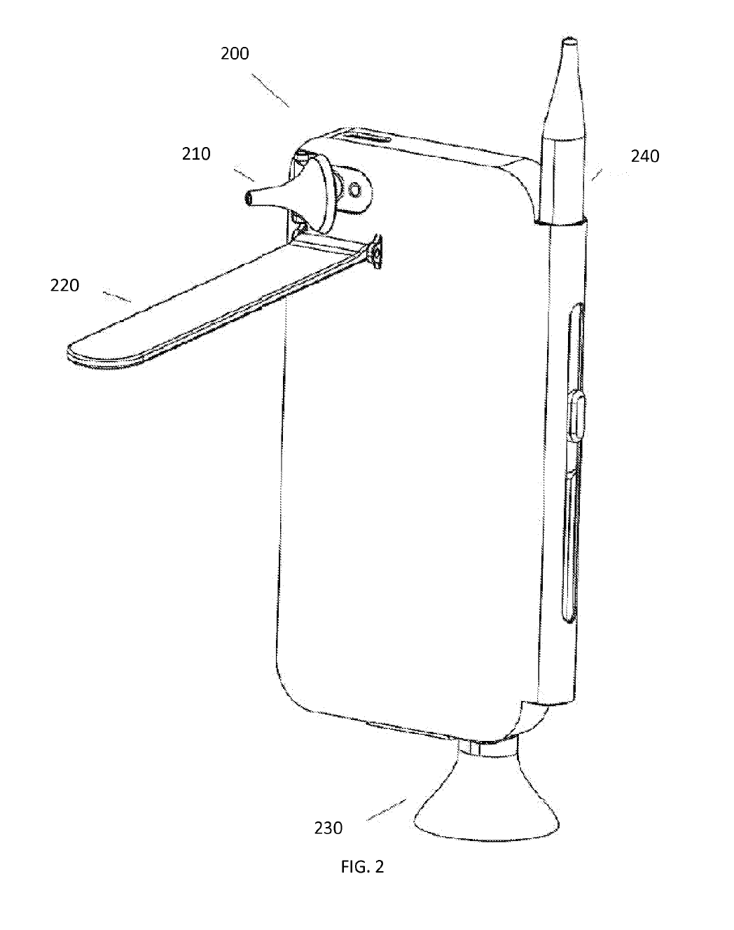

In another embodiment of the present invention shown in FIG. 2, it can be seen that an examination apparatus comprises a smartphone 10 snugly fitted into a removable all in one jacket 200 with various medical devices attached to it. The removable jacket 200 may contain a speculum 210 located in front of the camera of the smartphone 10 (said speculum 210 may contain an illumination means), a tongue depressor 220 adjacent to the camera of the smartphone 10.

The removable jacket 200 may also contain a thermometer 240. The thermometer 240 may be an integral part of the jacket 200, or alternatively the thermometer 240 might be a stand-alone thermometer (common household thermometer for example), snap connected to the jacket 200.

The removable jacket 200 may also contain a diaphragm 230 located in front and in close proximity to the microphone of the smartphone 10, wherein the sounds generated by the diaphragm 230 are converted by a microphone of the smartphone 10 into an electrical signal, which may be additionally amplified by a dedicated amplifier.

This arrangement allows the examination apparatus to be used for a Preliminary Examination which comprises an ear examination, a throat/mouth examination and an auscultation (listening to lung and heart sounds).

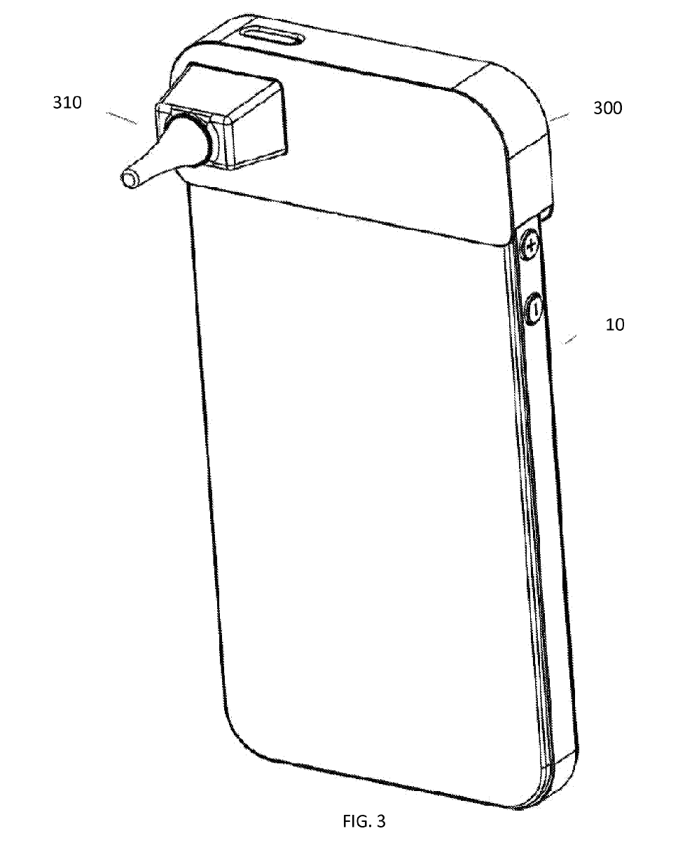

In yet another embodiment of the present invention shown in FIG. 3, a removable jacket 300 may contain only one medical device (for example a speculum, 310). Wherein only one dedicated medical device is attached at a time to the smartphone 10 and the examination apparatus is used for a specific checkup.

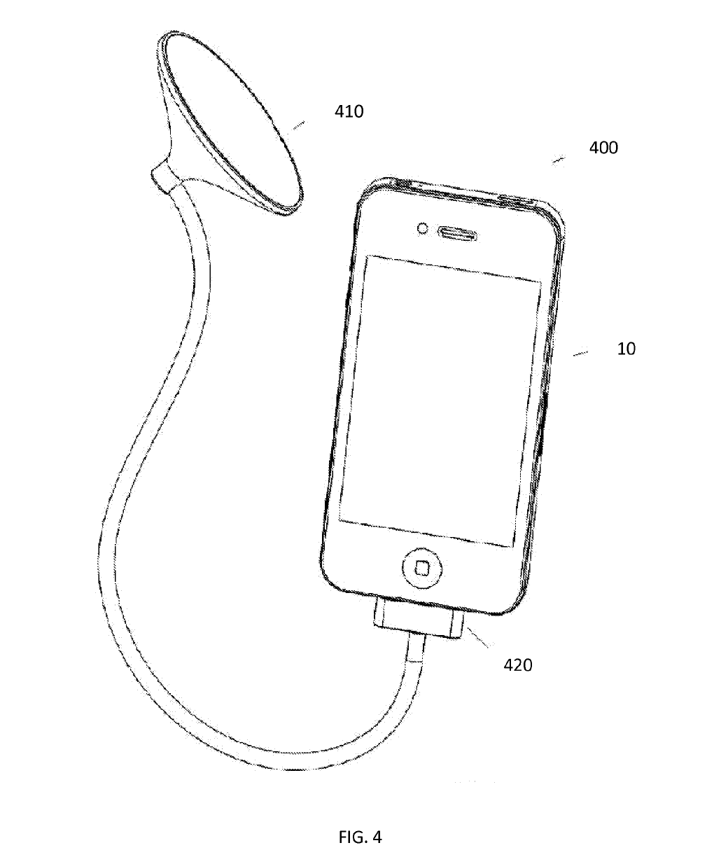

In yet another further embodiment of the present invention shown in FIG. 4, it can be seen that an examination apparatus 400 comprises a smartphone 10 and a medical device (diaphragm, 410) connected directly to the smartphone 10.

The diaphragm 410 may be connected to the microphone of the smartphone 10 by an acoustic link (such as hollow flexible tubing). The diaphragm 410 may also contain a microphone, wherein the microphone is electrically connected (via an amplifier, if needed) to the smartphone 10. Alternatively the diaphragm 410 may be connected to the smartphone via a dedicated detection circuit 420 which may contain a microphone and an amplifier. In another embodiment of the present invention, the connection between the diaphragm 410 and the smartphone 10 may be a wireless connection.

The connection of the diaphragm 210 to the smartphone 10, allows the examination apparatus 400 to be used as a stethoscope. The stethoscope can be utilized to pick up acoustic information from a fetus inside a pregnant patient. This information can be utilized to diagnose life signs and the condition of a fetus by the patient herself or by a doctor from a remote location.

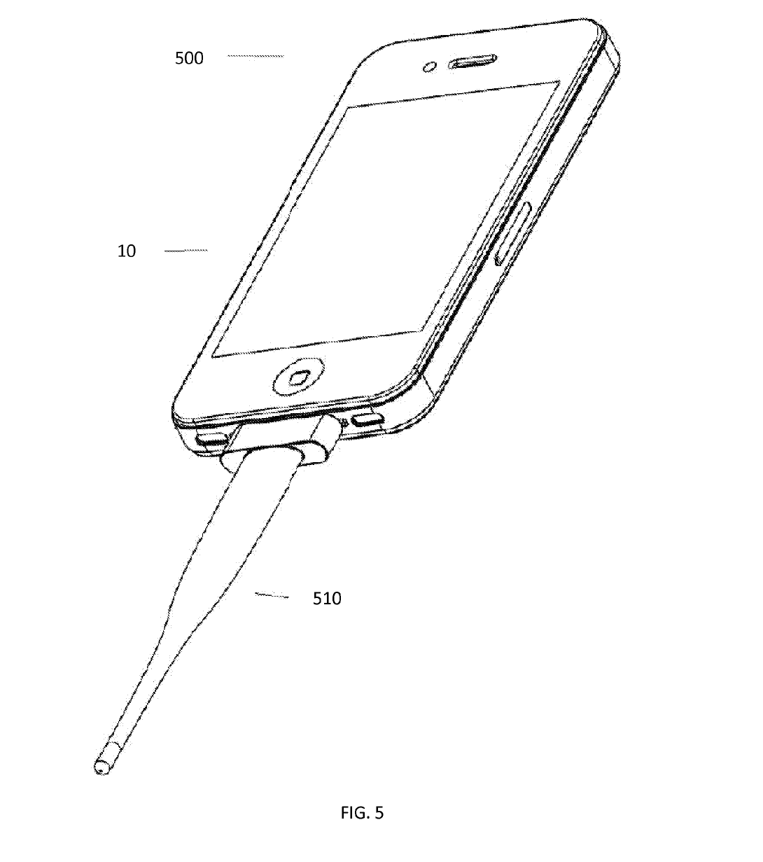

And in yet another further embodiment of the present invention shown in FIG. 5, it can be seen that an examination apparatus 500 comprises a smartphone 10 and a medical device (thermal sensor 510) connected directly to the smartphone 10. This allows the examination apparatus 500 to be used as a thermometer.

Although described as a Smart phone attachment, an all in one examination module and a removable (all in one) jacket can be operated with a tablet computer, laptop, desktop and etc.

The examination results (the examination results may be additionally processed by dedicated software) or examination itself is transmitted via wireless network (internet) in real time to the physician. The physician may further provide a set of instructions as of how to operate the device to obtain information which is adequate for analysis. Depending on the diagnosis set by the physician, the patient may receive a medical prescription which is automatically sent to the pharmacy or he might be instructed to visit a hospital/clinic for further treatment/observation.

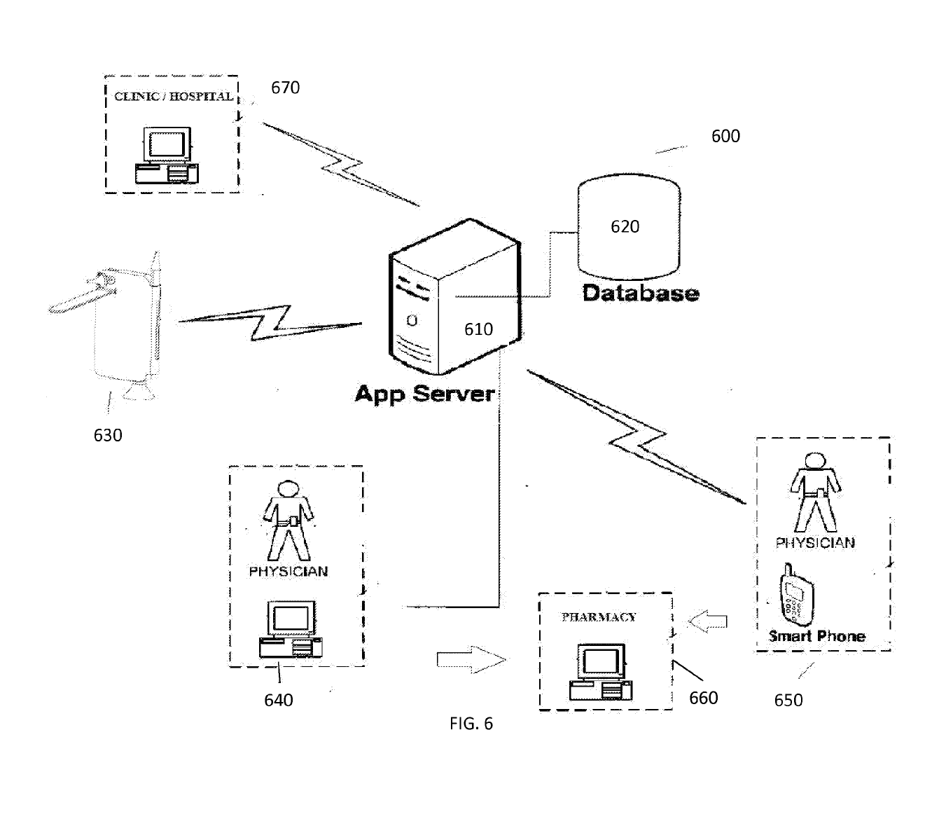

FIG. 6 shows a schematic diagram of a remote diagnosis system 600 of the present invention. The remote diagnosis system 600 comprises an examination apparatus 630 connected via a wireless network to a computer server 610. The server is also connected via wireless or cable based network to a physician 640, 650. The physician is able via the smartphone or the computer to observe the patient in real time and receive the examination result. The results of the examination and the diagnosis are reported to the patient and also stored in the medical history file in a database 620. Based on the results of the examination, the physician may prescribe a drug prescription and send it to the patient and/or pharmacy 660. Additionally, the physician may schedule a treatment/observation at the clinic/hospital 670 for the patient. The authentication of the patient is performed by matching the ID of his smartphone to the authorized smartphone registered by the system during the service registration process.

The present invention also comprises a method for remote examination and diagnosis. In this method, the physician examines the patient by use of an examination apparatus of the present invention. Based on the results of the examination, the physician prescribes a drug prescription or/and schedules a visit to the hospital or clinic for the patient. The examination results of each patient are securely stored in the database.

The retrieval of the information from the database and logging into the examination session is done by using a unique combination of username and password for each patient, as is well known in the art.

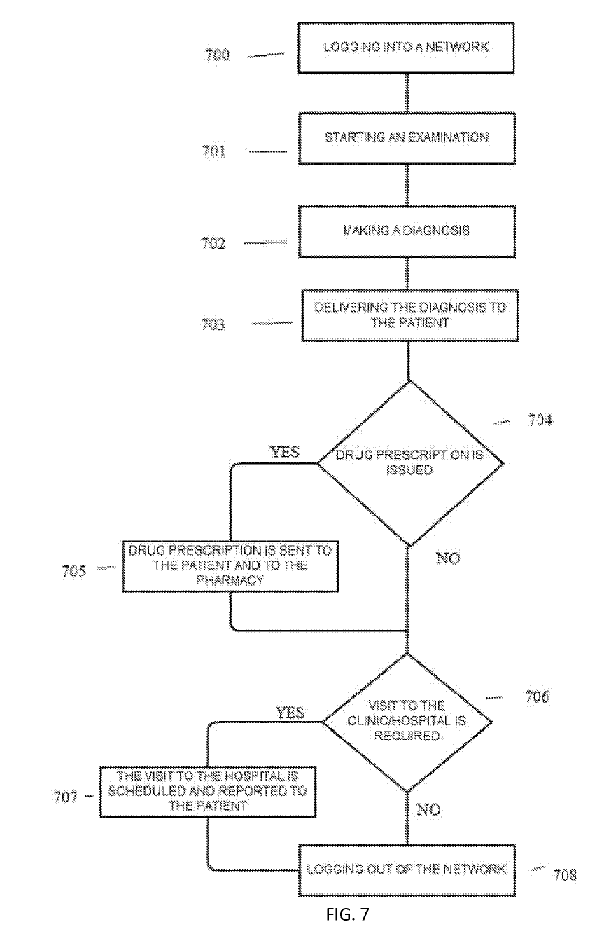

An exemplary method for remote examination and diagnosis is shown in the FIG. 7.

The method comprises the following steps:

Step 700: The operator (which may be a patient or any other person such as a parent of a child who is the patient, or a caregiver of a patient) logs in into a network to schedule a remote examination.

Step 701, starting the examination: The operator receives a call (3G video call) as scheduled by a physician. The video call can be performed over video communication applications such as Skype.

The patients overall look and sound is examined by the physician (this can be done by a regular 3G call without the device).

The operator receives instructions by the physician to attach the Preliminary Diagnosis attachment (all in one examination module or all in one jacket) to the smartphone.

Step 702: The physician makes a preliminary diagnosis via the device and instructs the patient and/or operator as of how to operate the device to obtain information which is adequate for analysis.

Step 703: The diagnosis is delivered to the patient, and is stored at the database.

Step 704: If needed, a drug prescription is issued by the physician.

Step 705: The drug prescription is sent to the patient and to the pharmacy and to the database.

Steps 706, 707: If needed, a visit to the hospital/clinic is scheduled and reported to the patient.

Step 708: The operator logs out of the network.

The method may also include crediting the patient each time a physical visit to the medical center has been avoided by utilizing the apparatus. The credit may include a discount rate, coupons, lottery tickets or any other sort of compensations.

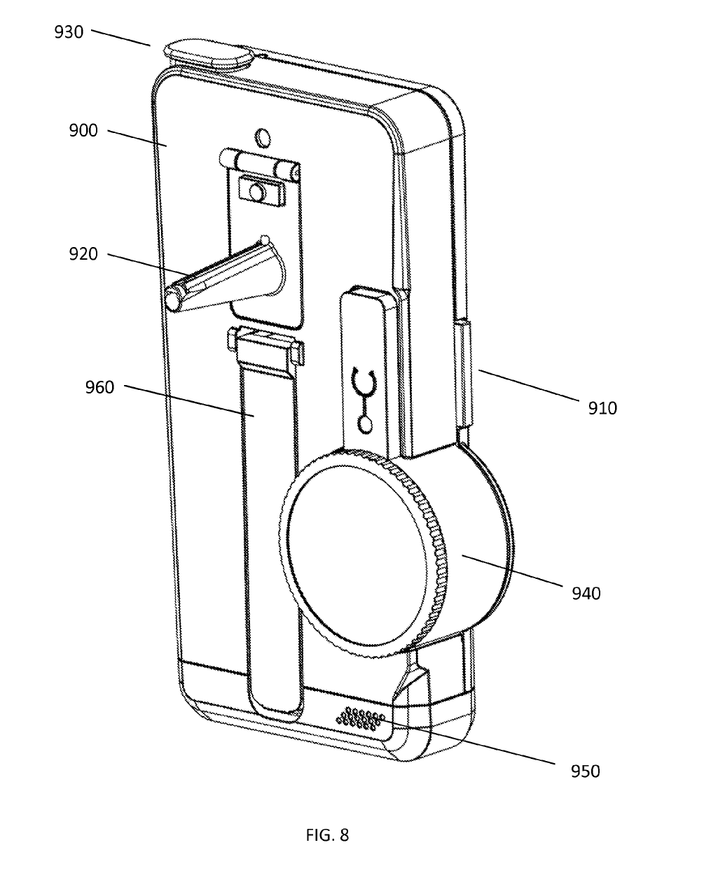

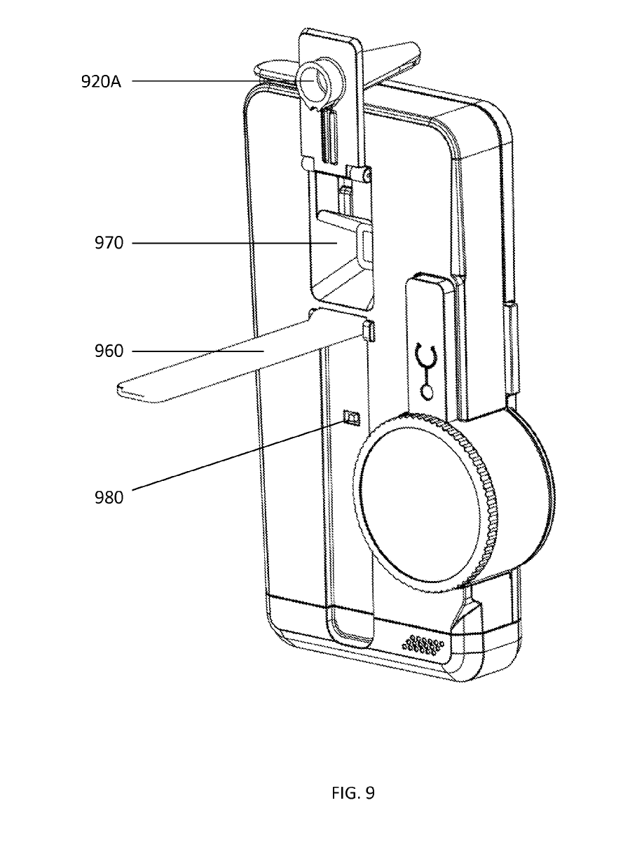

FIG. 8 and FIG. 9 schematically shows a perspective view of a jacket 900 configured to be attached to a standard smart phone for performing the preliminary remote medical examination. The jacket includes a hinge opening 910, when the jacket is opened the smart phone (not shown) is inserted into the jacket 900. The jacket 900 contains the sensors and the accessories adapted to pick up the required information for a remote preliminary diagnosis. For instance, a nozzle 920 is attached over the video camera of the smart phone (not shown). The nozzle 920 contains an ergonomic structure to securely be inserted into the ear of a patient, the nozzle 920 further incorporates a series of rod lenses and illumination means to ensure adequate image quality from inside the ear as further detailed in FIG. 9. The nozzle 920, can be removed or hinged aside 920A to utilize the video camera of the smart phone 970 while not performing the ear inspection. The jacket also includes a digital thermometer 930 for checking the body temperature of the patient, the thermometer 930 can be drawn in and out of the jacket 900 like a stylus. A stethoscope 940 is provided for listening to vital organs at the abdominal, chest and back area. The physician can guide the patient on line as to where to place the stethoscope 940 over the body. The stethoscope utilizes the microphone of the smartphone 950 to pick up audio signals without using a cord attached to the sensor which comes in contact with the body. A tongue depressor 960 can be utilized to provide a better view of the throat area. A switch 980 for turning illumination on or off is also provided.

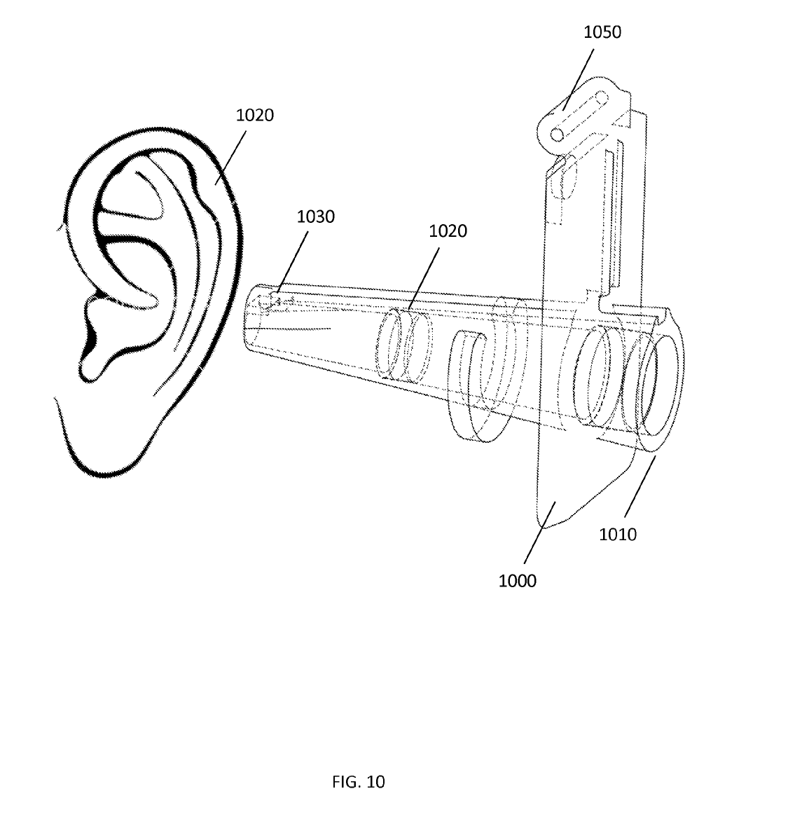

FIG. 10 schematically shows a perspective view of a nozzle 1010 designed to capture images inside the ear 1020. The nozzle incorporates a series of rod lenses and illumination means to illuminate the inspected area. The illumination can be performed by LEDs at the tip of the nozzle or by fiber optics which draw light from another source to the inspected area. Alternatively, integral illumination means of the smart phone can also be utilized to illuminate the inspected area. A protrusion is provided 1030 to restrict inadvertent insertion of the nozzle too deep into the ear. An integral battery for the jacket is also provided 1050.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features.

What is claimed is:

1. A medical examination apparatus for performing a set of remote examinations comprising:
a. a thermometer for checking body temperature;
b. a nozzle incorporating a lens ergonomically designed to be inserted into an ear to provide imaging inside the ear;
c. a stethoscope; and
d. an insert for a tongue depressor;

2. A medical examination apparatus according to claim 1, wherein the apparatus is a single jacket adapted to be attached to a smart phone.

3. A medical examination apparatus according to claim 1, wherein the apparatus is attached to a smart phone by a wired interface.

4. A medical examination apparatus according to claim 2, wherein a video camera, and a communication of the smart phone is utilized to pick up information from the apparatus and transmit it to a medical center.

5. A medical examination apparatus according to claim 3, wherein a video camera, and a communication of the smart phone is utilized to pick up information from the apparatus and transmit it to a medical center.

6. A medical examination apparatus according to claim 1, further comprising:
e. a video camera:
f. a transceiver; and
g. a battery;
wherein the medical examination device is self-contained.

7. A medical examination apparatus according to claim 6, wherein the information picked up by the apparatus is transmitted to a medical center.

8. A medical examination apparatus according to claim 6, further comprising;
h. an algorithm for analyzing the information picked up by the apparatus;
wherein the algorithms' analysis result is displayed to the patient.

9. A medical examination apparatus according to claim 1, wherein the physician can view on line the information picked up by the apparatus.

10. A medical examination apparatus according to claim 1, wherein the physician can remotely configure the parameters of the apparatus to obtain better information during the remote diagnosis.

11. A medical examination apparatus according to claim 1, wherein the apparatus is a kit of sensors configured to be attached to a smart phone.

12. A medical examination apparatus according to claim 1, wherein the information picked up by the apparatus is automatically filed and saved on an internet cloud.

13. A method for remote preliminary medical diagnosis comprising the following steps:
a. Attaching an apparatus with sensors to a smart phone;
b. sensing a patient's body temperature;
c. inspecting the internal of the ear for infections;
d. inspecting the patients throat for infections;
e. listening to the patients body via a stethoscope;
f. transmitting the acquired information to a remote physician;
wherein the physician analyses the acquired information and determines whether the patient needs to come to a medical center for further inspections.

14. A method for remote preliminary medical diagnosis according to claim 13, further comprising issuing a digital prescription according to the said analysis.

15. A method for remote preliminary medical diagnosis according to claim 13, further comprising a web based server for scheduling, filling, backing up and managing the remote medical diagnosis of multiple patients.

16. A method for remote preliminary medical diagnosis according to claim 13, further comprising an application running on a smartphone for presenting instructions and assisting the examination process.

17. An application according to claim 16, wherein the application is designed as a game to pacify children patients while the medical diagnosis is performed without disrupting the examination process.

18. A method for remote preliminary medical diagnosis according to claim 15, wherein the authentication of the patient is performed by recognizing details of the patients' smart phone.

19. A method for remote preliminary medical diagnosis according to claim 15, wherein the patient can access the files of his remote medical examination on-line by logging into the web based server by a user name and password.

20. A method for remote preliminary medical diagnosis according to claim 13, wherein the patient is credited each time a visit to the medical center is avoided by the remote preliminary medical analysis.
